(19) United States
(12) Patent Application Publication
(10) Pub. No.: US 2018/0071086 A1
(43) Pub. Date: Mar. 15, 2018

(54) Multiple Aperture Sling for Mitral Heart Valves

(71) Applicant: Infinity Patent Holdco LLC, Loveland, CO (US)

(72) Inventor: Ivan Vesely, Larkspur, CO (US)

(21) Appl. No.: 15/263,589

(22) Filed: Sep. 13, 2016

(51) Int. Cl. A61F 2/24

(52) U.S. Cl. CPC A61F 2/2409; A61F 2/243; A61F 2/2418; A61F 2220/0008; A61F 2220/0033; A61F 2230/001; A61F 2/246

(57) ABSTRACT

A sling with multiple openings may be installed in a mitral valve annulus. Within each opening, individual valves may be placed. The sling and valves may be delivered via catheter, and in some cases, the catheter size may be 18 French or smaller. The sling may have a cuff that may block otherwise open areas of the opening. The sling may be installed using commissural attachments that engage a mitral valve near the commissural point. The sling, valves, and cuff may be installed without stopping the heart, and because multiple valves are used, much smaller valves can be used, thereby reducing the catheter size.

### BACKGROUND

Mitral valve repair remains the gold standard therapy for all but the most severe form of mitral disease. Mitral valve dysfunction is either primary (degenerative, organic) or secondary (ischemic, dilated). Primary disease may be due to the valve itself, where the leaflets may be prolapsed and the annulus may be dilated. Secondary disease may be due to the ventricle, where the ventricle may be dilated and the leaflets pulled apart.

Mitral valve disease has been Alain Carpenter into three types. Type I may be where the annulus may be locally dilated. Type II may be where one or more leaflets are prolapsed. Type II may be where there may be ventricular dilation. Types I and II may be primary/generative/organic, while Type III may be secondary/ischemic.

Currently, there may be several least invasive approaches to treating Types I and II mitral valve disease. These include annular plication, stitches applied to the leaflets, and chord insertion. There is also total mitral valve implantation via catheter.

Total mitral valve implantation via catheter is a very complex technology and suffers from the risk of paravavular leakage, inadequate anchoring, and may be very bulky to be delivered via catheter.

### SUMMARY

A sling with multiple openings may be installed in a mitral valve annulus. Within each opening, individual valves may be placed. The sling and valves may be delivered via catheter, and in some cases, the catheter size may be 18 French or smaller. The sling may have a cuff that may block otherwise open areas of the opening. The sling may be installed using commissural attachments that engage a mitral valve near the commissural point. The sling, valves, and cuff may be installed without stopping the heart, and because multiple valves are used, much smaller valves can be used, thereby reducing the catheter size.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

### BRIEF DESCRIPTION OF THE DRAWINGS

In the drawings,

FIGS. 1A and 1B are a diagram illustrations of a human heart.

FIGS. 2A and 2B are a diagram illustrations of a human mitral valve anatomy, with FIG. 2B showing a diseased valve.

FIGS. 3A and 3B are diagram illustrations of embodiments of a commissural clamp in open and closed positions.

FIG. 4 is a diagram illustration of an embodiment showing clamps installed in the commissural regions.

FIGS. 5A, 5B, 5C, and 5D are a sequence of diagrams illustrating installation steps of a multi-valve mitral replacement.

FIG. 6 is a diagram illustration of an embodiment showing a pair of commissural clamps separated by a spacer balloon.

FIGS. 7A and 7B is diagram illustration of an embodiment showing a valve frame with a cuff

FIG. 8 is a diagram illustration of an embodiment showing curved commissural clamps.

FIG. 9 is a diagram illustration of an embodiment showing a large upper commissural clamp.

FIG. 10 is a diagram illustration of an embodiment showing a three valve replacement system.

### DETAILED DESCRIPTION

Multi Aperture Sling for Mitral Heart Valves

A multi-aperture sling for mitral heart valves may hold two, three, or more individual valves within the mitral opening. The sling, valves, and other components may be delivered via catheter as percutaneous transfemoral-transseptal implantation.

The sling may have openings that may receive prosthetic valves. By using two or more openings, the sling may allow for smaller valves to be used, which may reduce the largest catheter size for the installation procedure. Smaller catheters may allow for a wider range of patients to be treated, as well as be overall less invasive and have better patient recovery.

The sling and associated components consist of a set of clamps or other attachment mechanisms that may attach the sling to the heart in the area of the mitral valve. In one version, clamps or attachments are made in the commissural region of the mitral leaflets. In other versions, various claims, threaded attachments, staples, or other attachment mechanisms may be used. Some attachment mechanisms may involve piercing the heart valve muscle, while other attachment mechanisms may clamp to the leaflets, left atrium walls, or left ventricle walls.

Guide wires from the attachment mechanisms may be fed through attachment guides on the sling. The sling may be fed through a catheter into the left atrium, and the attachment guides may guide the sling into place. In some versions, the attachment mechanisms may have snap fits or other mechanical mechanism to fasten or attach the sling to the attachment mechanisms.

The sling may deploy within the left atrium with two or more annuluses. Each of the annuluses may be configured to accept a prosthetic valve. The valves may be delivered through a catheter, then expanded in an annulus to engage the sling's frame. Various mechanical mechanisms may be used to attach the valves to the frame.

The sling may support two, three, or more valves within the mitral annulus. Because the valves are much smaller than single prosthetic mitral valves, the valves may be delivered through smaller sized catheters.

A cuff may be attached to the sling. The cuff may block openings between the prosthetic valves and the left atrium wall, thereby minimizing profusion during a heartbeat. The cuff may be attached to the sling prior to installation, although some versions may have a separate cuff that may be installed after the sling's frame and may attach to the sling's frame. The cuff may have an exterior frame that may stretch the cuff membrane against the left atrium wall. Some versions may have attachment mechanisms for the cuff to be attached to the left atrium wall after or during installation.

Throughout this specification, like reference numbers signify the same elements throughout the description of the figures.

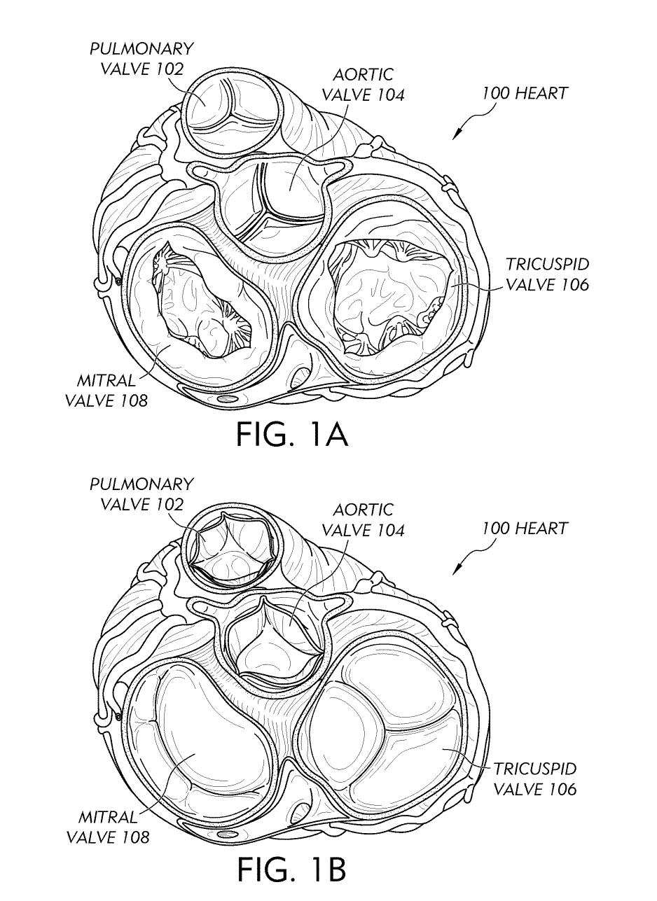

FIGS. 1A and 1B are diagram illustrations showing a section of a human heart 100, showing a pulmonary valve 102, the aortic valve 104, tricuspid valve 106, and mitral valve 108. FIG. 1A illustrates the tricuspid valve 106 and 108 in the open state, and the pulmonary valve 102 and aortic valve 104 in the closed state. FIG. 1B illustrates the tricuspid valve 106 and 108 in the closed state, and the pulmonary valve 102 and aortic valve 104 in the open state.

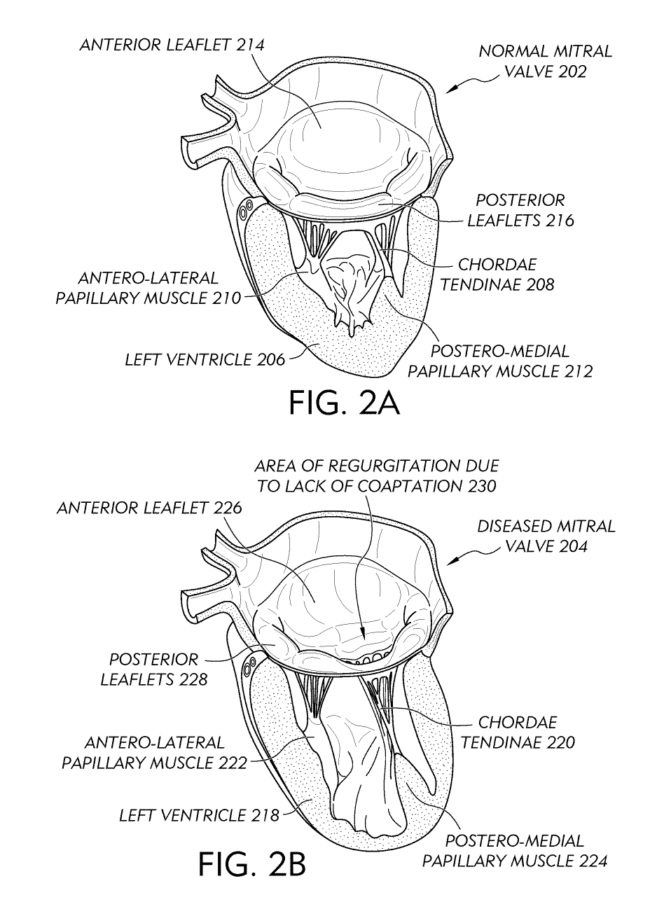

FIGS. 2A and 2B are diagram illustrations showing a mitral valve along with a section of the left ventricle. FIG. 2A illustrates a normal mitral valve 202, while FIG. 2B illustrates a diseased mitral valve 204.

FIG. 2A illustrates the left ventricle 206 and the chordae tendineae 208. The chordae tendineae 208 are actuated by the antero-lateral papillary muscle 210 and postero-medial papillary muscle 212.

The normal mitral valve 202 is illustrated with the anterior leaflet 214 and posterior leaflets 216 shown fully engaged with each other.

FIG. 2B illustrates the left ventricle 218 and the chordae tendineae 220. The chordae tendineae 220 are actuated by the antero-lateral papillary muscle 222 and postero-medial papillary muscle 224.

The diseased mitral valve 204 is illustrated with the anterior leaflet 226 and posterior leaflets 228 shown partially engaged, leaving an area of regurgitation 230 due to lack of coaptation of the leaflets.

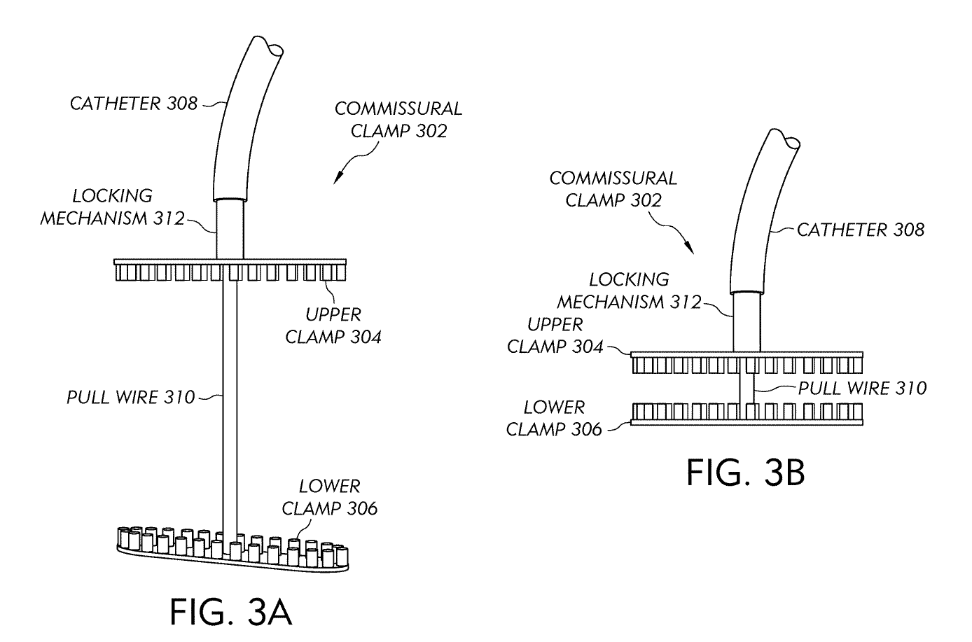

FIGS. 3A and 3B are diagram illustrations showing an embodiment of a commissural clamp 302. FIG. 3A illustrates the clamp 302 in an open position prior to locking, and FIG. 3B illustrates the clamp 302 in a closed or locked position.

The clamp has an upper clamp 304 and lower clamp 306 that may be delivered by a catheter 308. The catheter 308 may be a steerable catheter, or may be a catheter that may be itself be introduced through a larger, second steerable catheter.

The upper clamp 304 and lower clamp 306 may be attached to each other using a pull wire 310. The pull wire 310 may be used to cinch the lower clamp 306 and the upper clamp 304 together, such that a locking mechanism 312 may engage.

As will be seen in additional figures, the pull wire 310 may be used as a guide wire to guide a sling into place. The pull wire 310 may be detached after the sling may be installed.

The locking mechanism 312 may be any mechanism by which the upper clamp 304 and lower claim 306 may be locked together. A typical mechanism may be a snap or other mechanical lock.

The upper claim 304 and lower clamp 306 are illustrated with a set of serrations or teeth as an example of a mechanical engagement mechanism for the clamps. Other designs may have other mechanical designs to engage various anatomy of a patient's heart.

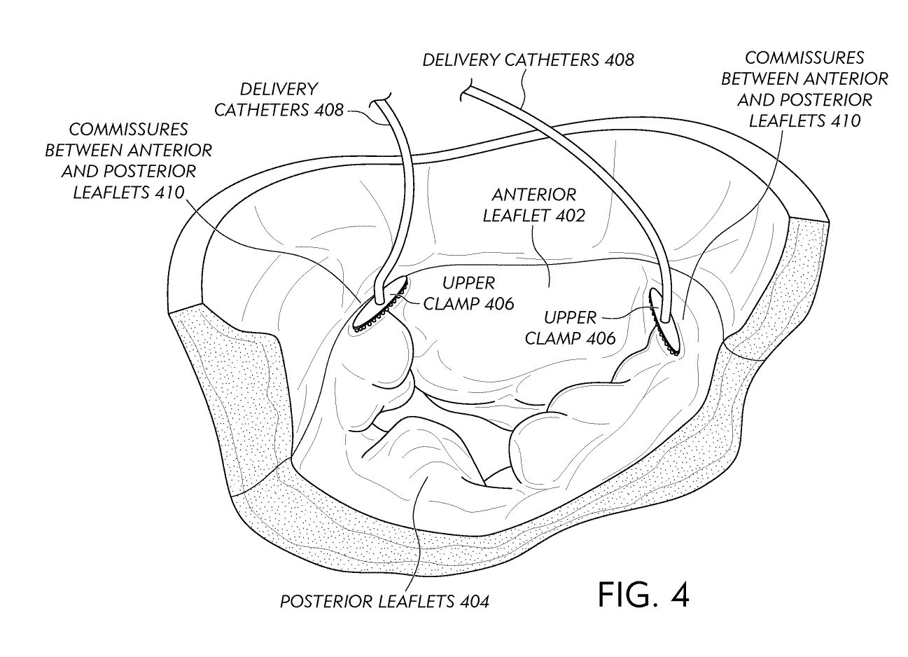

FIG. 4 is a diagram illustration showing clamps that may be installed in the commissural regions of a mitral valve. The anterior leaflet 402 and posterior leaflets 404 are illustrated with clamps 406 installed along with the delivery catheters 408. The clamps 406 are illustrated as cinching or gripping the leaflets at the commissures 410.

The clamps 406 may have an upper portion that may be visible above the leaflets and a lower portion that may not be visible in the current illustration. Each of the clamps 406 may engage a portion of the anterior leaflet 402 and the posterior leaflet 404. Such a position may provide a stable anchor for a sling into which two or more prosthetic valves may be installed.

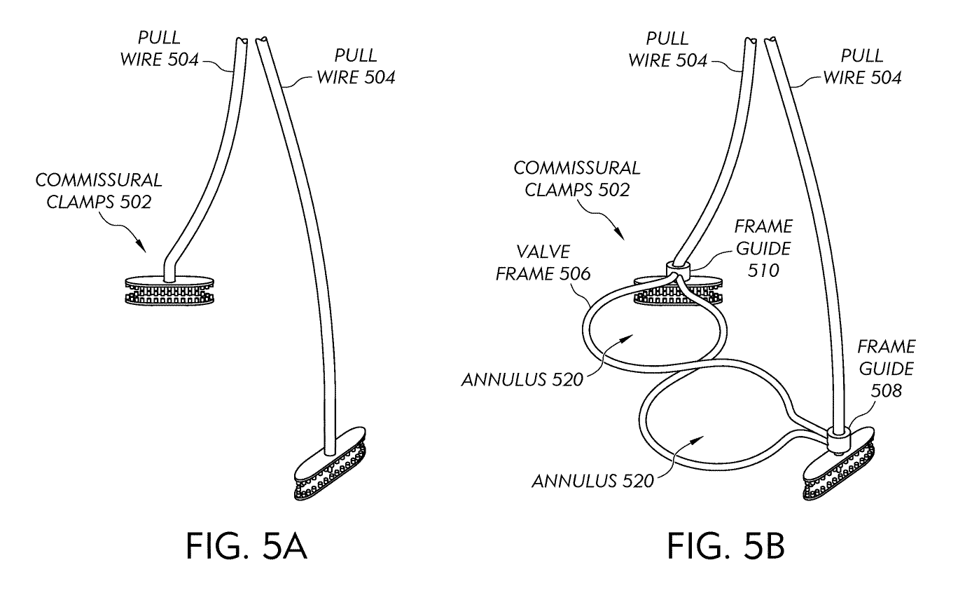

FIGS. 5A, 5B, 5C, and 5D may illustrate one sequence for installing a mitral valve replacement. The sequence is illustrated without showing the heart anatomy.

FIG. 5A illustrates the commissural clamps 502 along with their respective pull wires 504. The claims may be installed in the commissural region between the anterior and posterior leaflets of a mitral valve.

The pull wires 504 may be a wire attached to the clamps 502 and may be used for engaging and locking the upper and lower clamp components. The pull wires 504 may be left attached to the clamp components and used for delivery of other components as illustrated in later figures. The pull wires 504 may be detached after installation of the remaining components.

The clamps 502 may be delivered using a steerable catheter to place the clamps in the commissural region. The clamps may be opened such that the bottom clamp is placed below the leaflets and the upper clamp above the leaflets, then the clamps may be closed to capture the leaflets between the clamp portions. A locking mechanism may use a pull wire to pull against a delivery catheter to squeeze the clamps into position and engage a locking mechanism. After locking the clamp, the catheter may be retracted, leaving the pull wires in place for delivering some of the remaining components.

The clamps 502 may represent one attachment mechanism for a replacement valve system. Other attachment mechanisms may include other clamp designs, where various portions of the heart anatomy may be clamped. Such clamps may engage various portions of the heart, such as the walls of the left ventricle or left atrium. In some cases, a clamping system may engage a portion of the leaflets in addition to the walls of the left atrium or left ventricle. In other cases, a clamping system may engage only the leaflets. In still other cases, a clamping system may engage only the walls of the left atrium or left ventricle.

Other attachment mechanisms may penetrate the heart tissue in order to anchor an attachment. Such mechanisms may use pointed probes, threaded components, sutures, or other designs to penetrate a wall of the left atrium or left ventricle, and some may penetrate the leaflets. In some cases, an attachment mechanism may be made up of two, three, four, or more penetrations.

Such attachment mechanisms may be located at or near the commissural points, or may be located at other areas near the mitral valve. In some cases, attachment points may be distributed around the periphery of the mitral valve.

FIG. 5B illustrates the clamps 502 over which a valve frame 510 has been installed. The valve frame 510 may be guided in place with frame guides 510 and 512 that slide over the pull wires 504.

The valve frame 510 may be installed by routing the pull wires through the frame guides 510 and 512, then pushing the valve frame 510 through a catheter into the left atrium. The valve frame 510 may have a collapsed configuration for transport through the catheter, but may open to an expanded configuration when the valve frame 510 exits the catheter into the left atrium.

The valve frame 510 may collapse in various manners, depending on the valve frame design. In one manner, the valve frame 510 may be collapsed lengthwise so that the frame guides are proximal and distal along the catheter. In such a manner, the annuluses 520 may be collapsed and fed into a catheter. In another manner of collapsing the valve frame 510, the frame guides 510 may be brought together by bending the valve frame between the annuluses 520, then collapsing the annuluses 520 to fit inside a catheter.

The valve frame 510 may be made of spring material such that the valve frame 510 may expand to the configuration shown in FIG. 5B when unconstrained. In some cases, the valve frame 510 may be expanded into such a configuration by engaging various pull wires or other mechanisms that may apply mechanical force to change from a collapsed configuration to an expanded configuration.

The frame guides 508 and 510 may have a locking mechanism that may engage between the valve frame 506 and clamps 502. The locking mechanism may secure the valve frame 506 into place within the mitral annulus in preparation for installation of the remaining components.

FIG. 5C may illustrate a step where valves 512 and 514 may be installed into the annuluses 520 of the valve frame 506. The valves 512 and 514 may be valves that may be much smaller than a conventional replacement mitral valve, thereby allowing the valves and the remaining components to be delivered through smaller catheters than conventional replacement mitral valves.

In a typical design, a surgeon may use a steerable catheter to enter a patient's artery, enter the right atrium, then penetrate the septum to reach the left atrium. Once this larger catheter may be in place, the surgeon may install an attachment mechanism, such as the clamps 502, then the valve frame 506, and the valves 512 and 514.

The steerable catheter may be sized much smaller than a conventional replacement mitral valve, which is typically 34 French. The catheter used for installation of the multi-aperture system illustrated here may be 28 French, 26 French, 24 French, 22 French, 20 French, 18 French, 16 French, 14 French, or 12 French. The reduced size of the installation catheter may be primarily because the individual valves 512 and 514 may be much smaller and contain much less physical mass than a conventional, single replacement mitral valve.

The valves 512 and 514 may engage the valve frame 506 with an attachment mechanism. In many cases, the valves 512 and 514 may be configured with a mechanical engagement to the valve frame 506. The valves 512 and 514 may be individually installed into the valve frame 506. Each valve may be delivered through a catheter in a collapsed configuration, placed within an annulus in the valve frame 506, then opened up into an expanded configuration within the annulus. The valve may have a mechanical engagement mechanism to attach to the valve frame 506 and hold the valve in place.

FIG. 5D may illustrate a step where a cuff 516 may be delivered into the left atrium. The cuff 516 may block off otherwise open areas of the mitral annulus. The cuff 516 may have a cuff guide 524 that may be used to guide the cuff 516 into position as well as to lock the cuff 516 into place.

Cuff 516 is illustrated as a separate component that may be installed after the valve frame 506 may be installed. In some cases, the cuff 516 may be installed after the valve frame 506 and before the valves 512 and 514, while in other cases, the valve frame 506 may be installed, then the valves 512 and 514, then the cuff 516.

Some embodiments may combine the cuff 516 and the valve frame 506 into a single unit. Such embodiments may include the various components of the cuff 516 attached to the valve frame 506.

The cuff 516 may consist of a fabric or other membrane that may occlude openings within the mitral annulus. The cuff 516 may have a cuff frame 518, which may deploy upon entry into the left atrium, and may hold the membrane.

In some cases, the cuff 516 may have one or more attachment points along the periphery to allow attachment of the cuff membrane or cuff frame 518 to the heart anatomy. Such attachment points are not illustrated here. Such attachment points may include clamps, sutures, anchors, or other attachment mechanisms. Some such attachment mechanisms may clamp to the heart anatomy, while other attachment mechanisms may pierce a portion of the anatomy.

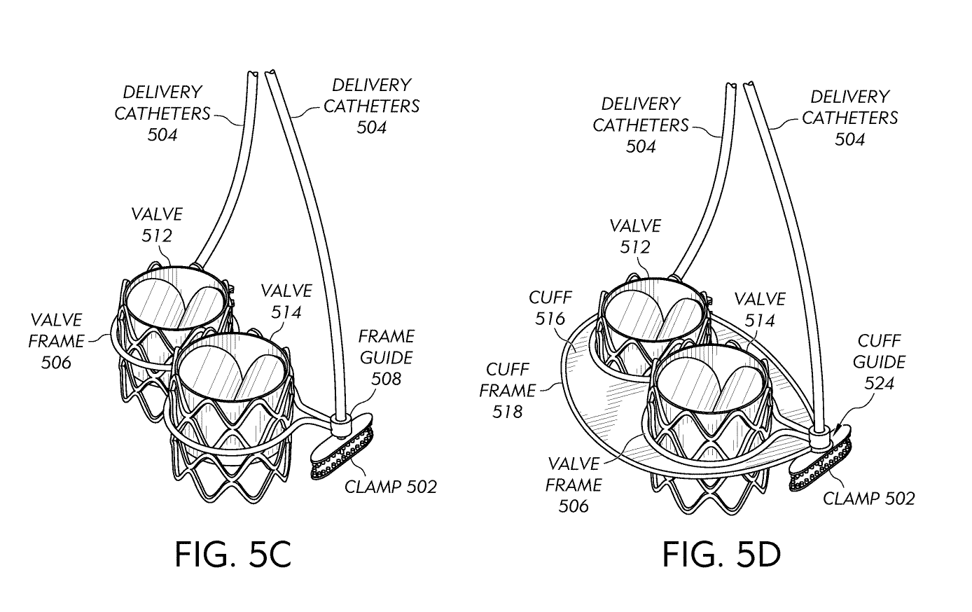

FIG. 6 is an illustration of a clamp installation system, showing catheters 602 and clamps 604 and 606. The clamps 604 and 606 are held apart with a spacer balloon 608. The spacer balloon 608 may force the clamps 604 and 606 into the commissural points between the posterior leaflet 601 and anterior leaflet 612.

The installation procedure for the clamps 604 and 606 may be to enter the left atrium, then deploy the spacer balloon 608. The spacer balloon 608 may be pressurized and help position the clamps 604 and 606 into the commissural areas, then a surgeon may affix the clamps 604 and 606 into place.

The spacer balloon 608 may be used to separate the clamps 604 and 606, while a surgeon positions one of the clamps and secures the clamp in place. The spacer balloon 608 may assist in positioning a second clamp after a first clamp is in place. In some cases, a first clamp may be installed prior to inflating the spacer balloon, which may be used to position the second clamp.

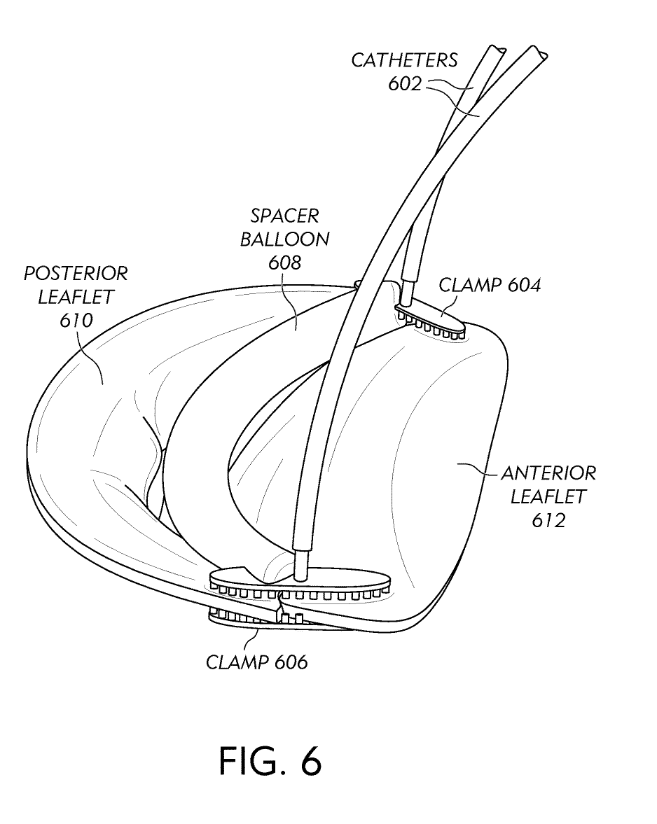

FIGS. 7A and 7B illustrate an embodiment with a smaller cuff that may not have a separate cuff frame. Pull wires 702 are shown connected to clamps 704. A valve frame 706 is illustrated as attached to the clamps 704.

Cuff 708 may be a fabric or other membrane that may be attached to the valve frame 706 between the annuluses of the valve frame 706. In the example, the cuff 708 may be attached to the valve frame 706 prior to installation.

FIG. 7B may show the assembly of FIG. 7A in position with a posterior leaflet 712. In such an installation, the valves 710 may fill most of the mitral annulus, causing the posterior leaflet 712 to be pushed into an open position, such that the valves 710 may operate in place of the normal anatomy.

The valves 710 may be positioned such that the leaflets of the valves 710 are at approximately the same height as the posterior leaflet 712. This may be accomplished by positioning the valve frame 706 and the mechanical attachments of the valves 710. The height of the valves 710 may be positioned higher or lower than the height of the posterior leaflets 712 in different cases.

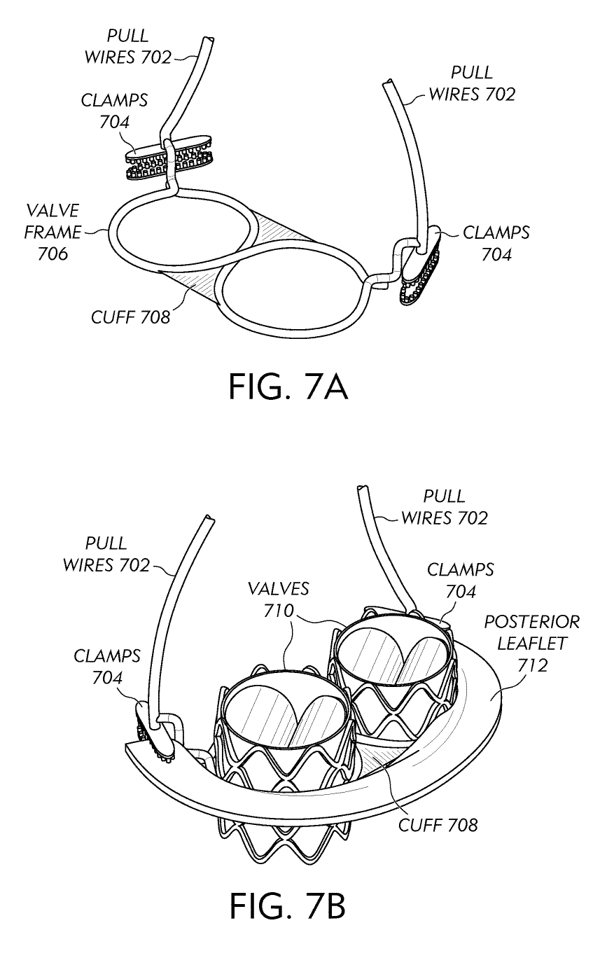

FIG. 8 is a top view illustration of a mitral valve 802. The anterior 804 and posterior 806 leaflets are shown, along with a set of clamps 808. The clamps 808 are illustrated as curved such that the clamps conform to the general anatomy while clamping the anterior 804 and posterior 806 leaflets.

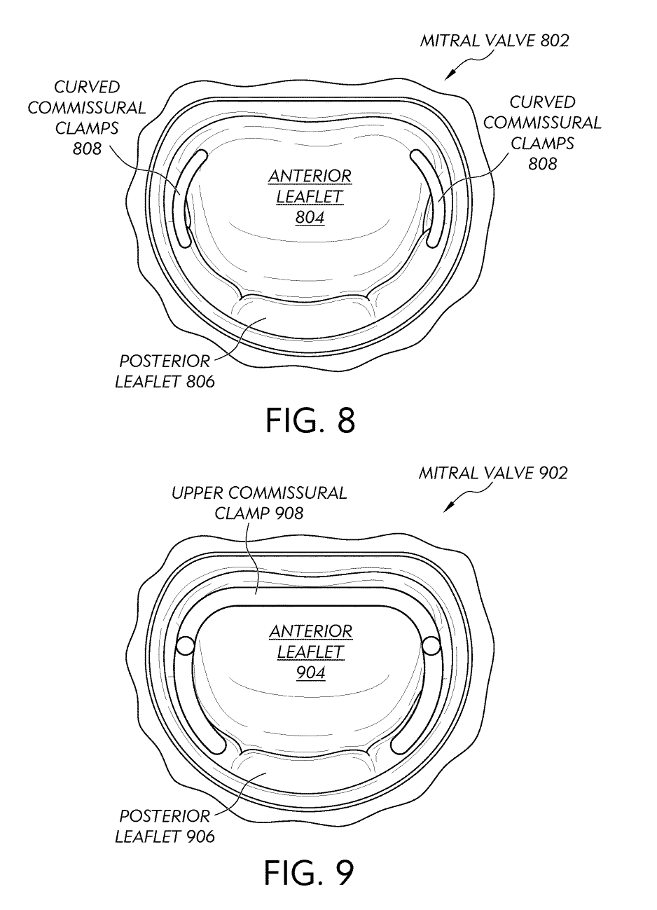

FIG. 9 is a top view illustration of a mitral valve 902. The anterior 904 and posterior 906 leaflets are show, along with a large upper commissural clamp 906. The upper clamp 906 may span the left atrium wall across the anterior leaflet 904 and may help the surgeon position the clamp 906 prior to attaching. The clamp 906 may have one, two, three, four, or more individual clamps or attachment mechanisms.

In some cases, the clamp 906 may have several barbs or other attachment mechanisms that may penetrate the leaflets or the heart wall. In other cases, the clamp 906 may have bottom clamps that may engage the leaflets at the commissural points.

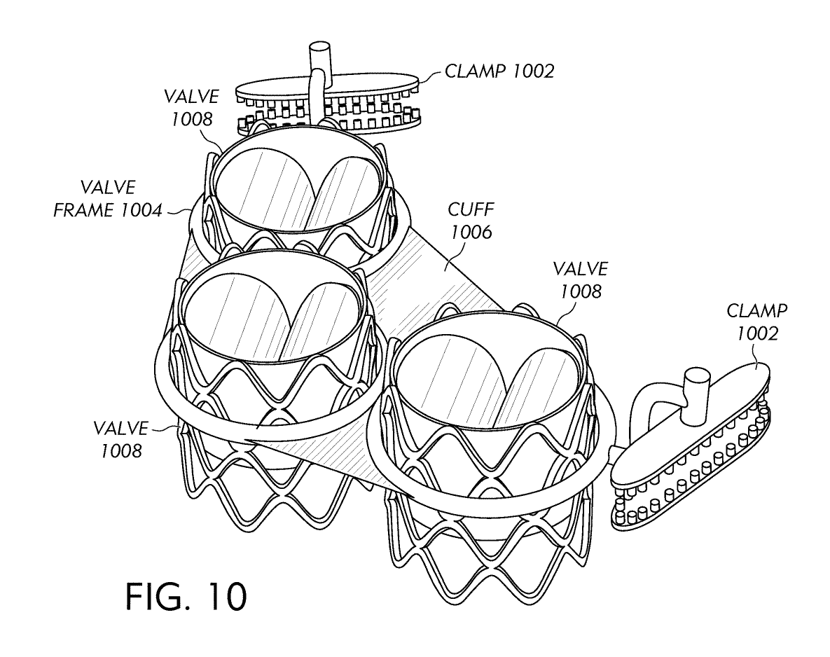

FIG. 10 is an illustration of a version of the system with three valves. The heart anatomy is not shown. Clamps 1002 are illustrated, along with a valve frame 1004.

The valve frame 1004 is illustrated with three annuluses, each of which may receive a valve 1008. A cuff 1006 is illustrated as having three components that serve to block the mitral annulus in the areas where the valves 1008 are not present.

The foregoing description of the subject matter has been presented for purposes of illustration and description. It is not intended to be exhaustive or to limit the subject matter to the precise form disclosed, and other modifications and variations may be possible in light of the above teachings. The embodiment was chosen and described in order to best explain the principles of the invention and its practical application to thereby enable others skilled in the art to best utilize the invention in various embodiments and various modifications as are suited to the particular use contemplated. It is intended that the appended claims be construed to include other alternative embodiments except insofar as limited by the prior art.

What is claimed is:

1. A sling for anchoring prosthetic mitral heart valves within a mitral annulus, said sling comprising:
a first anchoring attachment and a second anchoring attachment configured to be attached to a mitral valve annulus;
a first annulus having a first receptacle, said first receptacle configured to receive a first prosthetic valve;
a second annulus having a second receptacle, said second receptacle configured to receive a second prosthetic valve.

2. The sling of claim 1 further comprising:
a first anchoring attachment and a second anchoring attachment.

3. The sling of claim 2, said first anchoring attachment comprising a first guide wire receiver, said second anchoring attachment comprising a second guide wire receiver.

4. The sling of claim 3, said first anchoring attachment and said second anchoring attachment configured to attach to said mitral annulus at commissural points.

5. The sling of claim 3, said first anchoring attachment being attached to an anchor through a snap attachment.

6. The sling of claim 1 further comprising a cuff comprising at least one membrane and configured to at least partially block said mitral annulus.

7. The sling of claim 6, said membrane being a fabric membrane.

8. The sling of claim 6, said membrane having at least one attachment point, said attachment point configured to attach to heart tissue.

9. The sling of claim 8, said attachment point being an opening in said cuff.

10. The sling of claim 8, said attachment point being a portion of said cuff through which an anchor may be attached.

11. The sling of claim 6 further comprising a frame.

12. The sling of claim 11, said cuff being attached to said frame.

13. The sling of claim 11, said frame being configured to be installed in said mitral annulus prior to installing said cuff to said frame.

14. The sling of claim 11, said frame having a FIG. 8 component, said FIG. 8 component creating said first annulus and said second annulus.

15. The sling of claim 12, said FIG. 8 component further being attached to said first anchoring attachment and said second anchoring attachment.

16. The sling of claim 1 further comprising a third annulus having a third receptacle, said second receptacle configured to receive a third prosthetic valve.

17. The sling of claim 1, said first receptacle comprising a wire frame, said first prosthetic valve having an attachment mechanism configured to engage said wire frame.

18. The sling of claim 17 further comprising a cuff, said cuff having an exterior frame supporting one edge of said cuff.

19. The sling of claim 18, said cuff being further attached at least in part to said wire frame.

20. A delivery system for a mitral heart valve sling, said delivery system comprising:
said sling comprising:
a first annulus and a second annulus, said sling having a collapsed configuration and an expanded configuration;
said collapsed configuration comprising said first annulus and said second annulus in collapsed positions;
said expanded configuration comprising said first annulus and said second annulus in expanded configuration;
a first attachment guide and a second attachment guide;
a first member configured to constrain said sling, said sling being constrained in a collapsed position;
a first guide wire routed through said first attachment guide;
a second guide wire routed through said second attachment guide;
said delivery system being a catheter delivery system.

21. The delivery system of claim 20 having a catheter size no larger than 30 French.

22. The delivery system of claim 21 having a catheter size no larger than one of a group composed of:
28 French;
26 French;
24 French;
20 French;
18 French;
16 French;
14 French; and
12 French.

23. The delivery system of claim 20 further comprising:
a first anchor attached to said first guide wire; and
a second anchor attached to said second guide wire.

24. The delivery system of claim 23, said first anchor being a clamp having an open position and a closed position, said claim being adapted to clamp onto heart tissue in said closed position.

25. The delivery system of claim 24, said heart tissue comprising mitral valve leaflet tissue.

26. The delivery system of claim 24, said heart tissue comprising left ventricle tissue.

27. The delivery system of claim 24, said heart tissue comprising left atrium tissue.
